(19) United States
(12) Patent Application Publication
(10) Pub. No.: US 2026/0355480 A1
(43) Pub. Date: Nov. 12, 2026

(54) METAL CORES FOR GASTRIC RESIDENCE SYSTEMS

(71) Applicant: Nortiva Bio, Inc., Lexington, MA (US)

(72) Inventors: Ryan KOEPPEN, Watertown, MA (US); Tyler GRANT, Burlington, MA (US); Samuel CHADWICK, Acton, MA (US); David C. DUFOUR, Watertown, MA (US); Erik Robert Waldemar RYDE, Boston, MA (US)

(21) Appl. No.: 19/100,303

(22) PCT Filed: Aug. 3, 2023
(86) PCT No.: PCT/US2023/071617

§ 371 Date: Jan. 31, 2025

Related U.S. Application Data
(60) Provisional application No. 63/370,366, filed on Aug. 3, 2022.

(51) Int. Cl. A61M 31/00; A61K 47/02

(52) U.S. Cl. CPC A61M 31/002; A61K 47/02

(57) ABSTRACT

Provided are metal cores for gastric residence system and gastric residence systems comprising metal cores. A metal core according to some embodiments includes a plurality of arm mounts, each arm mount configured to receive an elongate arm of a gastric residence system, wherein the core is configured to bend into a compacted form such that each arm mount of the plurality of arm mounts approach each other and, when a plurality of elongate arms are attached to the metal core, the distal ends of the plurality of elongate arms approach each other, and wherein the metal of the metal core provides elasticity and shape memory to cause the metal core to unfold into an uncompacted form and, when a plurality of elongate arms are attached to the metal core, allow retention of the gastric residence system.

### CROSS-REFERENCE TO RELATED APPLICATIONS

This patent application claims priority benefit of U.S. Provisional Patent Application No. 63/370,366 filed Aug. 3, 2022. The entire contents of that patent application are hereby incorporated by reference herein.

### FIELD

The present disclosure relates to gastric residence systems and, more particularly, to gastric residence systems comprising metal cores.

### BACKGROUND

Gastric residence systems are delivery systems for agents which remain in the stomach for days to weeks, or even over longer periods, during which time drugs or other agents can elute from the systems for absorption in the gastrointestinal tract. Gastric residence systems are designed to be administered to the stomach of a patient, typically in a capsule which is swallowed or introduced into the stomach by an alternate method of administration (for example, feeding tube or gastric tube). Upon dissolution of the capsule in the stomach, the systems expand or unfold to a size which remains in the stomach and resists passage through the pyloric sphincter over the desired residence period (such as three days, seven days, two weeks, etc.). This requires mechanical stability over the desired residence period. Over the period of residence, the system releases an agent or agents, such as one or more drugs, preferably with minimal burst release, which requires careful selection of the carrier material for the agent in order to provide the desired release profile. While resident in the stomach, the system should not interfere with the normal passage of food or other gastric contents. The system should pass out of the stomach at the end of the desired residence time, and be readily eliminated from the patient. If the system prematurely passes from the stomach into the small intestine, it should not cause intestinal obstruction, and again should be readily eliminated from the patient. These characteristics require careful selection of the materials from which the system is constructed, and the dimensions and arrangement of the system.

### SUMMARY

Provided herein are gastric residence systems with cores comprising metal. Gastric residence systems described herein are designed to be administered to a patient in a compacted form. The compacted form may be achieved by bringing together distal ends of a plurality of arms of a stellate-shaped gastric residence form, causing the gastric residence system to bend at a core (from which the plurality of arms extend). When in the patient (e.g., within the stomach of the patient), the gastric residence system is configured to open from its compacted form to an open, or uncompacted form. However, when a gastric residence system is stored in its compacted form, its ability to return to its uncompacted form decreases over time due to material creep, particularly gastric residence systems comprising elastomeric cores. This creep minimizes the shelf life of the gastric residence system.

Accordingly, provided herein are gastric residence systems having cores comprising metal. As explained above, a stellate-shaped gastric residence system bends at a core to achieve a compacted form. Using metal in the core can extend the shelf life of the gastric residence systems, as metal does not creep as much as other materials used for gastric residence system cores, such as polymers.

The metal used within the cores of the gastric residence systems described herein can take any of several configurations and/or shapes. For example, in some embodiments, the metal component of a core can be formed using a metal wire and bent to form a stellate shape. The metal wire may comprise a number of “arms” or arm mounts that corresponds to a number of arms of the gastric residence system. (In other words, each arm of the gastric residence system may be attached to an “arm” of the stellate-shaped metal wire.) Such metal cores may be formed using laser cutting or stamping (e.g., in a flat metal core configuration) or using a jig (e.g., in a continuous wire core configuration). In some embodiments, overmolding may be used to attach the arms of the gastric residence system to the metal core. Overmolding may also be used to adhere polymers to the metal core to control the stiffness of the core, for example, and/or to coat the metal core to prevent the metal of the core coming in direct contact with a patient's body.

In some embodiments, provided is a gastric residence system, the gastric residence system comprising: a metal core, and a plurality of elongate arms, each elongate arm of the plurality of elongate arms comprising a proximal end connected to the metal core and a distal end, wherein the gastric residence system is configured to be folded into a compacted form by bending the metal core such that each arm mount of the plurality of arm mounts approach each other and the distal ends of the plurality of elongate arms approach each other, and wherein the metal of the metal core provides elasticity and shape memory to cause the gastric residence system to unfold into an uncompacted form and allow retention.

In some embodiments, provided is a gastric residence system, the gastric residence system comprising: a metal core having a plurality of arm mounts, and a plurality of elongate arms, each elongate arm of the plurality of elongate arms comprising a proximal end connected to an arm mount of the metal core and a distal end, wherein the gastric residence system is configured to be folded into a compacted form by bending the metal core such that each arm mount of the plurality of arm mounts approach each other and the distal ends of the plurality of elongate arms approach each other, and wherein the metal of the metal core provides elasticity and shape memory to cause the gastric residence system to unfold into an uncompacted form and allow retention. The number of elongate arms is equal to the number of arm mounts, so that each of the elongate arms is attached to a corresponding arm mount.

In some embodiments of the gastric residence system, the gastric residence system is configured to be stored in the compacted form for 30 days and then administered to a patient.

In some embodiments of the gastric residence system, the gastric residence system is configured to maintain the compacted form for 30 days and return to an uncompacted form such that the creep angle of the gastric residence form after the 30 days is within 3% of the creep angle before the 30 days.

In some embodiments of the gastric residence system, the metal core has a flat shape, such that a height and a width of the metal core is at least 10 times greater than a thickness of the metal core.

In some embodiments of the gastric residence system, a height of the metal core is the same as a width of the metal core.

In some embodiments of the gastric residence system, the metal core comprises a plurality of arm mounts, wherein each elongate arm of the gastric residence system is configured to attach to the metal core at an arm mount of the plurality of arm mounts.

In some embodiments of the gastric residence system, the metal core comprises nitinol. The nitinol can comprise ultra-low inclusion nitinol. The nitinol can comprise ultra-pure nitinol. The nitinol can comprise ultra-low inclusion, ultra-pure nitinol.

In some embodiments of the gastric residence system, each elongate arm of the plurality of elongate arms is overmolded onto the metal core.

In some embodiments of the gastric residence system, the gastric residence system comprises a degradable linker attached to an arm mount of the metal core and an elongate arm attached to the degradable linker.

In some embodiments of the gastric residence system, the metal core is formed using metal wire.

In some embodiments of the gastric residence system, the metal core is formed with stamped metal sheets.

In some embodiments of the gastric residence system, the metal wire has a cross-section that is from 0.01 to 0.02 inches in diameter (0.254 mm to 0.508 mm in diameter).

In some embodiments of the gastric residence system, the metal core is formed using flat sheets of metal.

In some embodiments of the gastric residence system, at least one elongate arm of the plurality of elongate arms comprises a therapeutic agent.

In some embodiments, a core for a gastric residence system is provided, the core comprising: a metal core having a plurality of arm mounts, each arm mount configured to receive an elongate arm of a gastric residence system, wherein the core is configured to bend into a compacted form such that each arm mount of the plurality of arm mounts approach each other and, when a plurality of elongate arms are attached to the metal core, the distal ends of the plurality of elongate arms approach each other, and wherein the metal of the metal core provides elasticity and shape memory to cause the metal core to unfold into an uncompacted form and, when a plurality of elongate arms are attached to the metal core, allow retention of the gastric residence system.

In some embodiments of the core, the metal core is configured to maintain the compacted form for 30 days and return to an uncompacted form such that the creep angle of the gastric residence form after the 30 days is within 3% of the creep angle before the 30 days.

In some embodiments of the core, the metal core has a flat shape, such that a height and a width of the metal core is at least 10 times greater than a thickness of the metal core.

In some embodiments of the core, a height of the metal core is the same as a width of the metal core.

In some embodiments of the core, the metal core is configured to maintain the compacted form for 30 days and return to an uncompacted form such that the creep angle of the metal core after the 30 days is within 3% of the creep angle before the 30 days.

In some embodiments of the core, the metal core comprises nitinol. The nitinol can comprise ultra-low inclusion nitinol. The nitinol can comprise ultra-pure nitinol. The nitinol can comprise ultra-low inclusion, ultra-pure nitinol.

In some embodiments of the core, each arm mount is configured to receive an elongate arm that is overmolded onto the arm mount.

In some embodiments of the core, each arm mount is configured to receive a degradable linker and an elongate arm is attached to the degradable linker.

In some embodiments of the core, the metal core is formed using metal wire.

In some embodiments of the core, the metal wire has a cross-section that is from 0.01 to 0.02 inches in diameter (0.254 mm to 0.508 mm in diameter).

In some embodiments of the core, the metal core is formed with stamped metal sheets.

In some embodiments of the core, at least one elongate arm of the plurality of elongate arms comprises a therapeutic agent.

In some embodiments, a method of manufacturing a metal core for a gastric residence system is provided, the method comprising: wrapping metal wire using a jig to form a metal core comprising a plurality of arm mounts.

In some embodiments of the method, wrapping the metal wire comprises wrapping the metal wire around a plurality of pins of a jig to form a circular-shaped metal form.

In some embodiments of the method, the method comprises advancing a plurality of inserts into the circular-shaped metal form to form a stellate-shaped metal form.

In some embodiments of the method, the method comprises heating the stellate-shaped metal form to generate a stellate-shaped metal core for a gastric residence system.

In some embodiments of the method, heating the stellate-shaped metal form comprises heating the stellate-shaped metal form to 500 °C or greater.

In some embodiments of the method, the metal wire comprises nitinol. The nitinol can comprise ultra-low inclusion nitinol. The nitinol can comprise ultra-pure nitinol. The nitinol can comprise ultra-low inclusion, ultra-pure nitinol.

In some embodiments of the method, the metal wire has a cross-section that is from 0.01 to 0.02 inches in diameter (0.254 mm to 0.508 mm in diameter).

In some embodiments of the method, the two ends of the metal wire are joined together.

In some embodiments of the method, the two ends of the metal wire are not joined together.

In some embodiments of the method, the stellate-shaped metal form is quenched after heating.

In some embodiments of the method, the metal core has a flat shape such that a height and a width of the metal core is at least 10 times greater than a thickness of the metal core.

In some embodiments of the method, a height of the metal core is the same as a width of the metal core.

In some embodiments of the method, the metal core is configured to be bent into a compacted form and maintain the compacted form for 30 days and return to an uncompacted form such that the creep angle of the metal core after the 30 days is within 3% of the creep angle before the 30 days.

In some embodiments, a method of manufacturing a metal core for a gastric residence system is provided, the method comprising: stamping a metal sheet to form a metal core comprising a plurality of arm mounts.

In some embodiments of the method, the metal sheet comprises nitinol. The nitinol can comprise ultra-low inclusion nitinol. The nitinol can comprise ultra-pure nitinol. The nitinol can comprise ultra-low inclusion, ultra-pure nitinol.

In some embodiments of the method, the metal core has a flat shape such that a height and a width of the metal core is at least 10 times greater than a thickness of the metal core.

In some embodiments of the method, a height of the metal core is the same as a width of the metal core.

In some embodiments of the method, the metal core is configured to be bent into a compacted form and maintain the compacted form for 30 days and return to an uncompacted form such that the creep angle of the metal core after the 30 days is within 3% of the creep angle before the 30 days.

In some embodiments, any one or more of the features, characteristics, or elements discussed above with respect to any of the embodiments may be incorporated into any of the other embodiments mentioned above or described elsewhere herein.

### BRIEF DESCRIPTION OF THE FIGURES

FIGS. 1A and 1B show different gastric residence system configurations, according to some embodiments;

FIG. 2 shows a gastric residence system in a folded, or compacted configuration, according to some embodiments;

FIGS. 3A-3D show various configurations of metal-comprising cores for a gastric residence system, according to some embodiments;

FIG. 4 shows a metal wire core, according to some embodiments;

FIG. 5 shows a detailed drawing of a metal wire core, according to some embodiments;

FIGS. 6A-6C show various views of one example of a continuous metal wire core, according to some embodiments;

FIGS. 7A-7D show various configurations of flat metal cores, according to some embodiments;

FIGS. 8A-8B shows a core of a conventional gastric residence system a core of a gastric residence system having a metal core, respectively, according to some embodiments;

FIGS. 9A-9B show metal cores with failures, according to some embodiments;

FIGS. 10A-10B show a top view of a gastric residence system comprising a metal core and a bottom view of a gastric residence system comprising a metal core, respectively, according to some embodiments;

FIGS. 11A-11C show a metal wire core being formed using a jig, according to some embodiments;

FIGS. 12A-12C show a process for forming a metal wire core using a jig, according to some embodiments;

FIGS. 13A-13C show various configurations for affixing an arm of a gastric residence system to a metal core, according to some embodiments;

FIG. 14 shows creep angle of a nitinol core compared to that of a polymeric (i.e., liquid silicone rubber core), according to some embodiments;

FIG. 15 shows the cyclic incubated non-planar compression test force over 2500 cycles for a nitinol core as compared to that of a polymeric core (i.e., liquid silicone rubber core), according to some embodiments;

FIG. 16 shows the non-planar compression test force for various nitinol core designs, according to some embodiments; and

FIGS. 17A-17E shows the various geometries of nitinol cores that are tested in FIG. 18. FIG. 17A and FIG. 17B show the geometries tested for Sheet #1 and Sheet #2. FIG. 17E shows the wire form shape used for Wire #1 and Wire #2 results.

FIG. 18 shows fatigue testing of various types of nitinol cores.

FIG. 19A shows a nitinol wire core with polymeric linkers affixed with an outer diameter of 12 mm (where the outer diameter is measured from the edge of one outermost linker to the outermost edge of the opposite linker assembly). FIG. 19B shows a nitinol wire core with polymeric linkers affixed with an outer diameter of 15 mm (where the outer diameter is measured from the edge of one outermost linker to the outermost edge of the opposite linker assembly).

FIG. 20 shows fatigue tests for the cores shown in FIG. 19A and FIG. 19B.

FIG. 21 shows a view of a nitinol wire core with polymeric linkers affixed.

FIG. 22 shows a cross-sectional view of the nitinol wire core of FIG. 21.

FIG. 23 shows another cross-sectional view of the nitinol wire core of FIG. 21.

FIG. 24 shows another view of the nitinol wire core of FIG. 21 where the components covered by the polymeric linkers (i.e., the hidden components) are shown with dashed lines.

### DETAILED DESCRIPTION

Provided herein are gastric residence systems having cores comprising metal. The gastric residence systems described herein include a core from which a plurality of arms extend. The arms comprise a proximal end that connects to the core, and a distal end opposing the proximal end and extending away from the core. To administer to a patient, the gastric residence system must assume a compacted form. To do this, the distal ends of the gastric residence system are brought together, forcing the core, where the proximal ends of the arms are connected, to bend. When the gastric residence system is held in its compacted form for an extended period of time, the material of the core creeps, or experiences permanent deformation. This permanent deformation prevents the gastric residence system from assuming its open, uncompacted form when administered to a patient.

Accordingly, the gastric residence systems provided herein comprise cores having metal. The metal of the gastric residence system cores allow the gastric residence system to have a longer shelf life (i.e., while in a compacted form) and still be able to resume its uncompacted form when administered to a patient. The metal does not experience creep, or permanent deformation as quickly as many polymeric materials, and thus, allow the gastric residence system to be shelf stable in its compacted form for a longer period of time relative to gastric residence systems having polymeric-only cores.

Discussed below are general principles of gastric residence systems, followed by a detailed discussion of the metal cores, nitinol cores specifically, methods of manufacturing metal cores, and examples.

### General Principles of Gastric Residence Systems

Provided below is a description of gastric residence systems and how they operate to deliver a therapeutic agent to a patient. In particular, the discussion includes a general description of how gastric residence systems are designed to deliver a therapeutic agent to a patient over an extended period of time, how gastric residence systems are configured for administration, how gastric residence systems are configured to deploy and deliver a therapeutic agent to the stomach of a patient, how the therapeutic agent of a gastric residence system elutes from the device such that the therapeutic agent is delivered to the patient, how the gastric residence system passes through the stomach, and how gastric residence systems are designed to account for some specific safety measures.

Gastric residence dosage forms can be designed to be administered to the stomach of a patient by swallowing, by feeding tube, by gastric tube, etc. Once a gastric residence dosage form is in place in the stomach, it can remain in the stomach for a desired residence time (e.g., three days, seven days, two weeks, etc.). A gastric residence dosage form that is properly in place in a stomach will resist passage through the pyloric valve, which separates the stomach from the small intestine. Gastric residence dosage forms can release a therapeutic agent (i.e., API or drug) over the period of residence with controlled release. While residing in the stomach, the dosage form may not interfere with the normal passage of food or other gastric contents. Once the desired residence time has expired, the dosage form passes out of the stomach (i.e., through the pyloric valve) and is readily eliminated from the patient.

To administer a gastric residence system to a patient, the gastric residence system can be folded into a form small enough to be swallowed or otherwise administered. In some embodiments, the folded gastric residence system is retained in a capsule or other container which can be swallowed by the patient. In some cases, the gastric residence system may be delivered to a patient via gastrostomy tube, feeding tube, gastric tube, or other route of administration to the stomach. Examples of folding and encapsulating the gastric residence system are provided in further detail below.

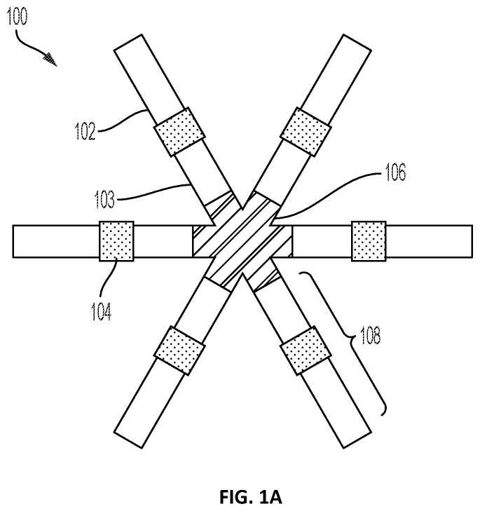

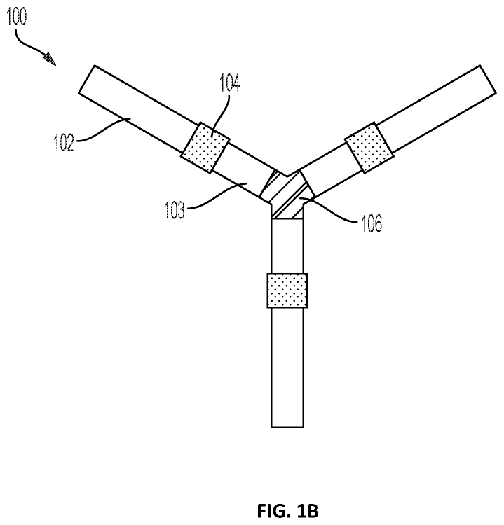

FIGS. 1A and 1B provide embodiments of foldable or compactable gastric residence systems. Specifically, the foldable or compactable gastric residence systems shown in FIGS. 1A and 1B are provided in an unfolded (or uncompacted) configuration.

As shown in FIG. 1A, gastric residence system 100 may be star-shaped (stellate) according to some embodiments. In some embodiments, a star-shaped gastric residence system 100 is constructed around core 106. Core 106 may include one or more elongate member 108, or “arms,” projecting radially. The arms may be formed by carrier polymer-agent components 102 and 103 and linker regions 104 comprising coupling polymer. Core 106 enables gastric residence system 100 to be folded for packaging into a capsule. Once the capsule dissolves in the stomach, gastric residence system 100 unfolds to the circular shape of its open, or unfolded, configuration, preventing passage through the pyloric valve.

While the linker regions 104 are shown as slightly larger in diameter than the segments 102 in FIG. 1A, they can be the same diameter as the segments, so that the entire arm 102-104-103 has a smooth outer surface.

In some embodiments, the stellate system may have an arm composed of only one segment, which is attached to the central elastomer by a linker region. This corresponds to FIG. 1A with the segments 103 omitted. The single-segment arms comprising segments 102 are then directly attached to central elastomer 106 via the linkers 104. The linkers can comprise a coupling polymer or a disintegrating matrix.

FIG. 1B shows gastric residence system 100 comprising three “arms” according to some embodiments. This configuration can also include core 106 from which the three “arms” radially extend. Each of the three arms comprise polymer-agent components 102 and 103 and linker region 104 as well.

A stellate system can be described as a gastric residence system for administration to the stomach of a patient, comprising a core, and at least one carrier polymer-agent component comprising a carrier polymer and an agent or a salt thereof, attached to the core, wherein each of the plurality of carrier polymer-agent components is an arm comprising a proximal end, a distal end, and an outer surface therebetween; wherein the proximal end of each arm is attached to the core and projects radially from the core, each arm having its distal end not attached to the core and located at a larger radial distance from the core than the proximal end; wherein each arm independently comprises one or more segments, each segment comprising a proximal end, a distal end, and an outer surface therebetween. In some embodiments, when two or more segments are present in an arm, each segment is attached to an adjacent segment via a linker region. In some embodiments, when two or more segments are present in an arm, one segment is directly attached to the other segment, without using a linker region. The linker region can be a coupling polymer or a disintegrating matrix. The arms can be attached to the core via a coupling polymer or a disintegrating matrix, and can have intervening portions of interfacing polymers. For the plurality of at least three arms, or for a plurality of arms, a preferred number of arms is six, but three, four, five, seven, eight, nine, or ten arms can be used. The arms should be equally spaced around the central elastomer; if there are N arms, there will be an angle of about 360/N degrees between neighboring arms.

The coupling polymers of the gastric residence system, which serve as linker regions, are designed to break down gradually in a controlled manner during the residence period of the system in the stomach. In some embodiments, a linker region may completely dissolve, allowing the components connected the linker region to completely separate. In some embodiments, the linker region may not completely dissolve, but break down such that the components connected to the linker region remain attached. However, in this case, the linker regions may break down sufficiently to compromise the structural integrity of the gastric residence system such that it can pass through the pyloric valve and through the intestines. If the gastric residence system passes prematurely into the small intestine in an intact form, the system is designed to break down much more rapidly to avoid intestinal obstruction. This is readily accomplished by using enteric polymers as coupling polymers. Enteric polymers are relatively resistant to the acidic pH levels encountered in the stomach, but dissolve at the higher pH levels found in the duodenum. Use of enteric coupling polymers as safety elements protects against undesired passage of the intact gastric residence system into the small intestine. In the system shown in FIG. 1A, at least the coupling polymer used for the couplings 104 are made from such enteric polymers.

In additional embodiments, a time-dependent coupling polymer or linker can be used. Such a time-dependent coupling polymer or linker degrades in a predictable, time-dependent manner. In some embodiments, the degradation of the time-dependent coupling polymer or linker may not be affected by the varying pH of the gastrointestinal system.

In additional embodiments, different types of linkers can be used in the gastric residence systems. That is, both enteric linkers (or enteric coupling polymers) and time-dependent linkers (or time-dependent coupling polymers) can be used. In some embodiments, a single multi-segment arm of a stellate system can use both an enteric linker at some linker regions between segments, and a time-dependent linker at other linker regions between segments.

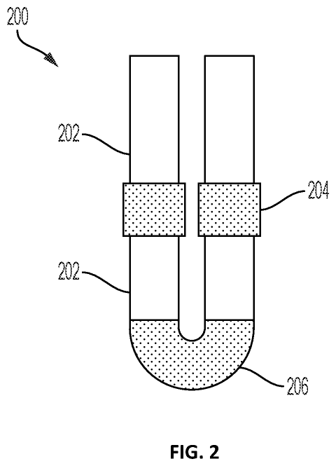

FIG. 2 shows folded gastric residence system 200 according to some embodiments. As shown, the device can fold at core 206, bringing the ends of each “arm” together. The Figure also shows how the carrier polymer-agent components 102 and couplings 104 of each arm may be oriented in a folded configuration.

The folded configuration of gastric residence system 200 can be bound (i.e., held in a folded configuration) with a sleeve or band. In some embodiments, a gastric residence system in a folded configuration (with or without a sleeve or band) may be encapsulated with a capsule to form a gastric residence dosage form. In some embodiments, the gastric residence dosage form may be coated with a reverse-enteric coating to ensure deployment of the gastric residence system in a patient's stomach.

Once the gastric residence dosage form arrives in the stomach of the patient, the capsule and/or capsule coating of the gastric residence dosage form may dissolve/open and release the folded gastric residence system. Upon release, the gastric residence system unfolds to assume an open configuration, such as a ring shape or a star shape as provided in FIGS. 1A and 1B. The dimensions of the open gastric residence system are suitable to prevent passage of the device through the pyloric valve for the period of time during which the device is to reside in the stomach. In some embodiments, the folded gastric residence system can also be secured by a dissolvable retaining band or sleeve that can prevent premature deployment of the gastric residence system in case of a failure of the capsule.

While in the stomach, the gastric residence system is compatible with digestion and other normal functioning of the stomach or gastrointestinal tract. The gastric residence system does not interfere with or impede the passage of chyme (partially digested food) or other gastric contents which exit the stomach through the pyloric valve into the duodenum.

Once released from the capsule into the stomach, the therapeutic agent of the gastric residence system begins to take effect. In some embodiments, the gastric residence system comprises a plurality of carrier polymer-agent components. The carrier polymer-agent components may comprise a carrier polymer, a dispersant, and a therapeutic agent (or a salt thereof). The plurality of carrier polymer-agent components are linked together by one or more coupling polymer components. The therapeutic agent may be eluted from the carrier polymer-agent components into the gastric fluid of the patient over the desired residence time of the system. Release of the therapeutic agent is controlled by appropriate formulation of the carrier polymer-agent components, including by the use of the dispersant in formulation of the carrier polymer-agent components, and by milling of the therapeutic agent to particles of desired size prior to blending the agent with the carrier polymer and dispersant.

In some embodiments, a gastric residence system may include a filament (or “webbing”) between arms of a gastric residence system. Gastric residence systems having a filament may help improve the gastric residence of the gastric residence system. Specifically, a filament can help provide a more consistent gastric residence time and/or a longer gastric residence time. Thus, gastric residence systems that include a filament may provide more predictable and/or controllable gastric residence times. Gastric residence systems having predictable and/or controllable gastric residence times can minimize the possibility of the gastric residence system passing through the stomach and unfolding later in the gastrointestinal tract (i.e., intestine), or passing through the gastrointestinal tract without unfolding at all. In each of these possible scenarios, the therapeutic agent of the gastric residence dosage form is not delivered to the patient as intended.

In some embodiments, a filament wrapped circumferentially around a gastric residence system and connecting the arms of the gastric residence system, for example, can help prevent premature passage through a patient's gastric pylorus.

Additionally, coatings can be applied to outer surfaces of the gastric residence system. The coatings can include additional therapeutic agents or agents that can affect the release of therapeutic agents or the residence duration of the gastric residence system.

Once the desired residence time has expired, the gastric residence system passes out of the stomach. To do so, various components of the gastric delivery system are designed to weaken and degrade. The specific dimensions of the system are also taken into consideration. In its intact, open configuration, the gastric residence system is designed to resist passage through the pyloric valve. However, coupling polymer components of the gastric residence system are chosen such that they gradually degrade over the specified residence period in the stomach. When the coupling polymer components are sufficiently weakened by degradation, the gastric residence system loses critical resilience to compression or size reduction and can break apart into smaller pieces. The reduced-size dosage form and any smaller pieces are designed to pass through the pyloric valve. The system then passes through the intestines and is eliminated from the patient. In some embodiments, a gastric residence system may be made of soft material such that the gastric residence system can pass through a pyloric valve intact once the residence time expires without degrading into numerous smaller pieces.

There are some safety considerations to account for during the design and administration of the gastric residence system and the gastric residence dosage form. In particular, it is important that the gastric residence system remain in its folded configuration until it reaches the stomach. If the gastric residence system opens or unfolds prior to reaching the patient's stomach, the patient risks an esophageal obstruction. Similarly, if an intact gastric residence dosage form passes through the pyloric valve without opening and the gastric residence system expanding into its open configuration, there is a risk that it could do so in the patient's intestine, resulting in an intestinal obstruction. Accordingly, capsules and capsule coatings according to embodiments described herein have been designed to control the deployment of gastric residence systems for improved patient safety.

Examples of gastric residence systems may be found in PCT/US2018/051816, WO 2015/191920, WO 2017/070612, WO 2017/100367, WO 2018/064630, WO 2017/205844, WO 2018/227147, and U.S. 62/933,211, each of which is incorporated herein in its entirety.

### Metal Cores

As explained above, gastric residence systems provided herein bend at their core to assume a folded or compacted form. To increase shelf stability and decrease permanent deformation of the core over time as the compacted gastric residence system is held in its compacted form, gastric residence systems provided herein include cores comprising metal. In some embodiments, the core of a gastric residence system comprises only metal. In some embodiments, the metal comprises nitinol. In some embodiments, the metal comprises ultra-low inclusion nitinol. In some embodiments, the metal comprises ultra-pure nitinol. In some embodiments, the metal comprises ultra-low inclusion, ultra-pure nitinol. In some embodiments, the metal comprises spring steel, copper, or any alloy having shape memory and/or superelastic properties. In some embodiments, the core of a gastric residence system comprises metal and a polymeric material. For example, some embodiments may include a polymeric material overmolded on a metal component. The overmolded polymeric material may help control stiffness of the core and/or it may coat a portion of the metal component to minimize the amount of the metal component that comes in direct contact with the patient's body.

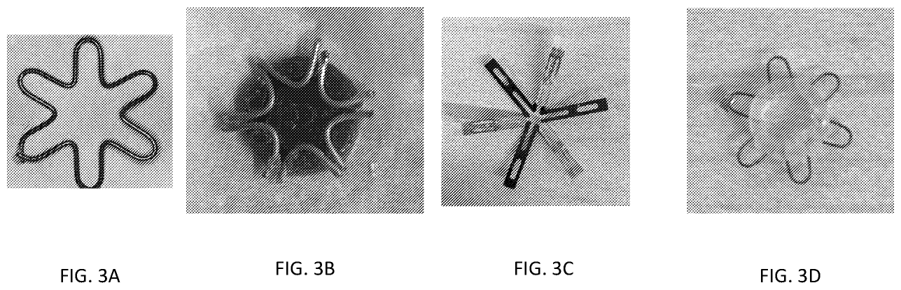

FIGS. 3A-3D show various configurations of metal-comprising cores for a gastric residence system, according to some embodiments. As shown, FIG. 3A shows a core made of metal wire and formed into a stellate shape to correspond to a stellate-shaped gastric residence system, according to some embodiments. A metal wire core may be prepared by molding the metal wire into a desired shape (e.g., a stellate shape). An arm of a gastric residence system may be affixed to each “arm” of the stellate-shaped metal core. In some embodiments, the two ends of the metal wire may be joined, to form a continuous metal wire (e.g., by crimping or welding). In some embodiments, the two ends of the metal wire may be left open. The opening may be positioned at an “arm” of the metal core such that it can be sealed by an arm of the gastric residence system when the arms are affixed to the metal core.

FIG. 3B shows a metal wire core having pointed “arms”. This type of metal core might be manufactured using a piecewise process that includes forming several pieces of the core separately, and connecting the pieces together to form the metal wire core. Each of the arms of a gastric residence system could be affixed to a pointed “arm” of the metal wire core.

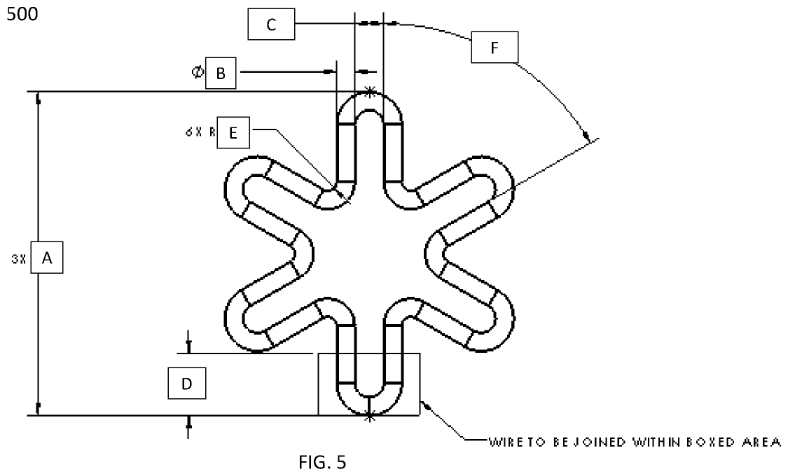

FIG. 3C shows a flat metal core, according to some embodiments. A flat metal core may be prepared using laser cutting or stamping. As shown in FIG. 3B as well as in FIGS. 5A-5D, a flat metal core may take any of a variety of shapes. In some embodiments, a flat metal core may be stellate-shaped like the gastric residence system. As explained with reference to FIG. 3A, each arm of the gastric residence system may be affixed to an “arm” of the stellate-shaped flat metal core.

FIG. 3D shows a metal wire core having a polymer overmold, according to some embodiments. Overmolding the metal core may help achieve optimal core stiffness. In some embodiments, overmolding the metal core may help minimize the amount of metal that is exposed to the patient's body. As shown, the polymer overmold may only cover a portion of the metal core. In some embodiments, the polymer may cover the entire metal core. In some embodiments, the combination of the polymer overmold and the arms of the gastric residence system may cover the entire metal core (though neither the polymer overmold nor the arms of the gastric residence system cover the entire metal core alone).

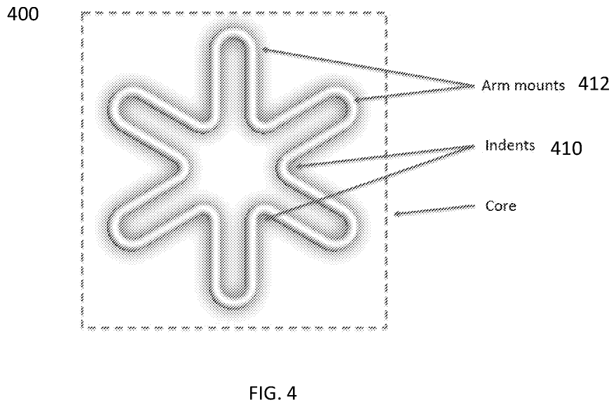

FIG. 4 shows a metal wire core 400 according to some embodiments. As shown, metal wire core 400 includes indents 410 and arm mounts 412.

FIG. 5 shows a detailed drawing of a metal wire core 500, according to some embodiments. As shown, a metal wire core 500 may be stellate-shaped to correspond to a stellate-shaped gastric residence system. Here, the stellate-shaped metal wire core includes six “arms”, each of which can receive an arm of the gastric residence system. In some embodiments, an arm of the gastric residence system may be overmolded onto the distal end of the arm of the metal wire core. In some embodiments, two ends of the wire used to form the metal wire core may be positioned within the overmolded area, such that they are embedded within the arm of the gastric residence system. By covering the ends of the wire with an arm of the gastric residence system, the potential for internal damage to a patient caused by the wire ends is eliminated.

The metal wire core 500 of FIG. 5 includes the following marked measurements: (A) diameter; (B) wire thickness; (C) distance between two wire sides of an arm (as measured between an interior surface of the two wire sides of the arm; (D) area within wire ends can be joined; (E) inner radius; and (F) angle between two adjacent arms.

The metal wire core 500 of FIG. 5 shows six “arms” to correspond to six arms of a gastric residence system, but a metal core (and gastric residence system) can have two, three, four, five, seven, eight, nine, ten, eleven, or twelve or more arms.

Exemplary measurements (in millimeters) are shown in FIG. 5. These measurements can apply to metal wire cores (as depicted) or to flat metal cores. In some embodiments, a metal core may have a diameter (marked as A in the figure and measured between an external surface of opposing arms such that the diameter includes the thickness of the wire) of 8-14 mm. In some embodiments, the metal core may have a diameter of less than or equal to 14, 13, 12, or 11 mm. In some embodiments the metal core may have a diameter of greater than or equal to 10, 11, 12, or 13 mm. In some embodiments, when the metal core is manufactured with metal wire, the metal wire may have a thickness (marked as B) of 0.1-1 mm. In some embodiments, the thickness of the metal wire may be less than or equal to 1, 0.8, 0.6, 0.4, or 0.2 mm. In some embodiments, the thickness of the metal wire may be greater than or equal to 0.1, 0.2, 0.4, 0.6, or 0.8 mm. In some embodiments, the metal core may have an internal radius (marked as E and as measured between two opposing rounded vertices or mouths, indents) of 0.25-2 mm. In some embodiments, the metal core may have an internal radius of less than or equal to 2, 1.5, 1, 0.75, or 0.5 mm. In some embodiments, the metal core may have an internal radius of greater than or equal to 0.25, 0.5. 0.75. 1, or 1.5 mm.

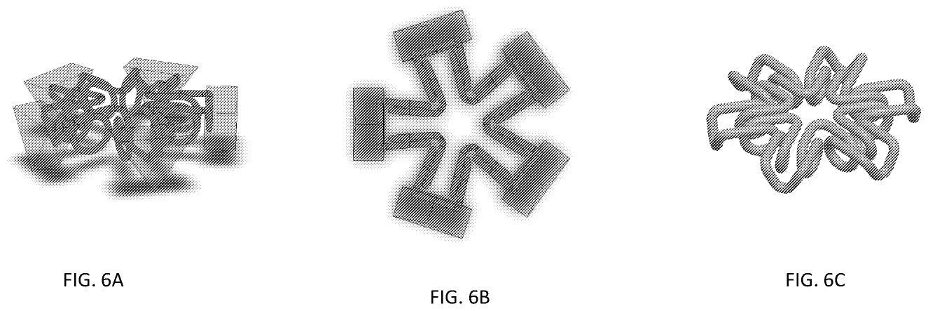

FIGS. 6A-6C show various views of one example of a continuous metal wire core, according to some embodiments. This particular embodiment uses springs to help the gastric residence system assume an uncompacted form. FIG. 6A shows a perspective view of a metal core with portions of arms of a gastric residence system overmolded onto each arm of the metal core. FIG. 6B shows a top view of a metal core with portions of arms of a gastric residence system overmolded onto each arm of the metal core. FIG. 6C shows a metal core without any arms of a gastric residence system overmolded onto the metal core.

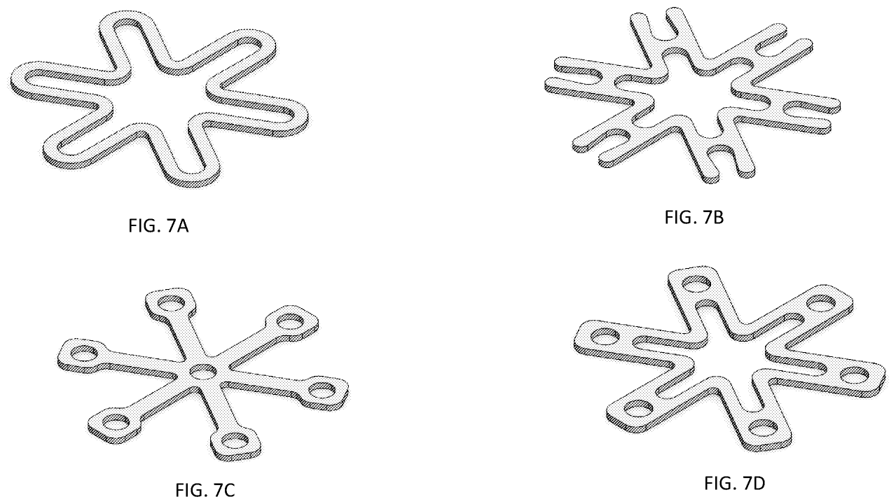

FIGS. 7A-7D show various configurations of flat metal cores (or metal “sheet” cores), according to some embodiments, a simpler geometry that may be easily adapted to a stamping process for high volume manufacturing. FIG. 7B shows a design having a “pronged” end effector that may offer advantages for adhering material molded or overmolded to it. FIG. 7C shows a design that deforms differently than the other designs, and its performance may be easier to predict. Finally, FIG. 7D shows a design having an alternative, simpler end effector design than FIG. 7B, but it may be more difficult to manufacture.

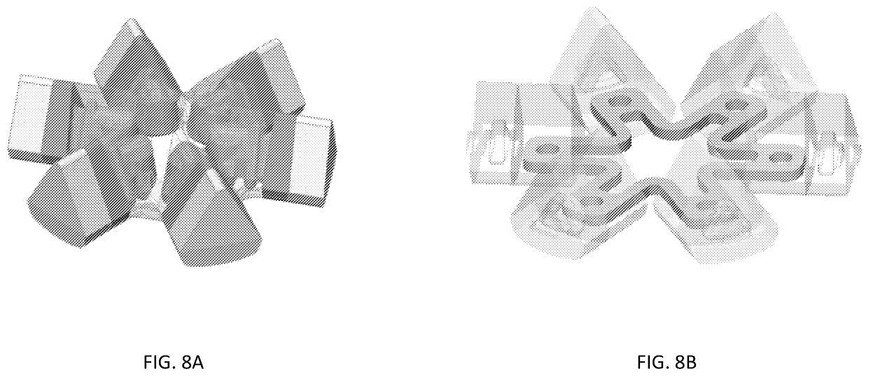

FIG. 8A shows a core of a conventional gastric residence system, without any metal. FIG. 8B shows a core of a gastric residence system having metal to reinforce and improve the performance properties of the gastric residence system (e.g., improve the gastric residence system's ability to assume an uncompacted form after maintaining a compacted form for an extended period of time).

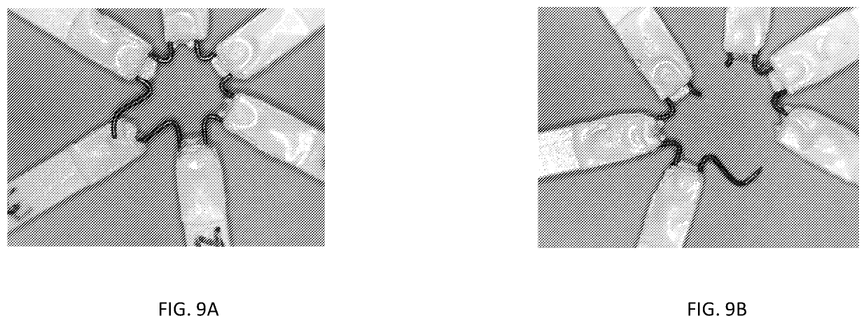

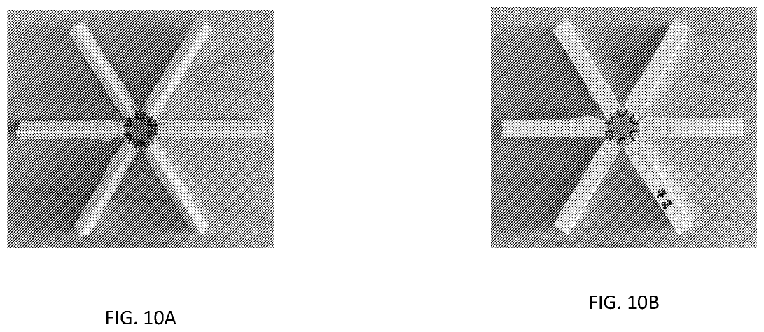

FIGS. 9A and 9B show some common obstacles with metal cores. Specifically, FIG. 9A shows that a wire end of a metal wire core may pop out of an affixed gastric residence system arm. FIG. 9B shows that a metal wire may break due to the bending of the metal core. These failures may occur during encapsulation (and/or when assuming an open/uncompacted configuration from a compacted configuration), while in a patient's stomach, or during handling. Thus, the form, materials, and specific manufacturing process for the metal cores disclosed here and the entire gastric residence system is important to minimize the possibility of such failures.

FIG. 10A shows a top view of a gastric residence system comprising a metal core, according to some embodiments, and FIG. 10B shows a bottom view of a gastric residence system comprising a metal core, according to some embodiments.

As used herein, the shelf life of a gastric residence system may be defined as the length of time that a gastric residence system may be stored in a compacted form without losing its ability to assume an uncompacted form. In some embodiments, a gastric residence system may be said to successfully assume an uncompacted form if the creep angle of the gastric residence system does not decrease more than 0-10, 0-5, or 0-2 percent over the course of the encapsulation (or shelf life) period. In some embodiments, the gastric residence system successfully assumes an uncompacted form if the creep angle of the gastric residence system does not decrease more than or equal to 10, 8, 6, 5, 4, 3, 2, 1, or 0.5 percent over the course of the encapsulation (or shelf life) period. In some embodiments, the gastric residence system successfully assumes an uncompacted form if the creep angle of the gastric residence system does not decrease less than or equal to 0, 0.5, 1, 2, 3, 4, 5, 6, or 8 percent over the course of the encapsulation (or shelf life) period.

In some embodiments, gastric residence systems comprising a metal core according to embodiments described herein may have a shelf life of 1 day to 10 years, 1 day to 5 years, 1 day to 1 year, 1-30 days, 30-90 days, or 90-180 days. In some embodiments, gastric residence systems comprising a metal core according to embodiments described herein may have a shelf life of less than or equal to 10 years, 8 years, 5 years, 3 years, 2.5 years, 2 years, 1.5 years, 1 year, 350 days, 300 days, 270 days, 240 days, 210 days, 180 days, 150 days, 120 days, 90 days, 60 days, 30 days, 20 days, 10 days, or 5 days. In some embodiments, gastric residence systems comprising a metal core according to embodiments described herein may have a shelf life of greater than or equal to 1 day, 5 days, 10 days, 20 days, 30 days, 60 days, 90 days, 120 days, 150 days, 180 days, 210 days, 240 days, 270 days, 300 days, 350 days, 1 year, 1.5 years, 2 years, 2.5 years, 3 years, 5 years, or 8 years.

### Nitinol Cores

In some embodiments, the metal cores may be made of nitinol, which is a metal alloy of nickel and titanium. Nitinol has exceptional shape memory and superelasticity properties. Nitinol can deform 10-30 times as much as ordinary metals and still return to its original shape. Whether nitinol behaves with shape memory or super elasticity depends on whether the material is above (superelasticity) or below (shape memory) its transformation temperature. Thus, gastric residence systems having metal cores comprising nitinol may be able to fold into a compacted form (at the nitinol core) and later, after remaining in a compacted form for a period of time (e.g., encapsulated), the gastric residence system may be able to return to its planar uncompacted form. Gastric residence systems comprising nitinol cores may be able to return to their original, uncompacted form, more readily and more easily than cores of other materials, and particularly, cores comprising only polymeric materials.

The nitinol cores described herein are based on the “austenite” phase nitinol, which has the superelastic properties as described above. The shape memory properties are typically associated with “martensite” phase nitinol, which permanently deforms very easily and will return to its original shape only when heated.

In some embodiments, a metal core made of nitinol is first formed (e.g., by wrapping nitinol wire around a jig, described in detail below) and heated to 500 °C or greater. Heating and quenching the formed nitinol shape sets the shape of the nitinol core. Thus, when the nitinol core is bent (i.e., when the gastric residence system is folded into a compacted form) and subsequently released from the bent shape (i.e., when a folded, encapsulated gastric residence system is released in a human body upon administration), the nitinol core can return to its originally formed shape, allowing the gastric residence system to effectively deliver a therapeutic agent to the patient.

In some embodiments, the nitinol used for the metal core is ultra-low inclusion nitinol. The ultra-low inclusion nitinol has a maximum 12.0 micron inclusion size and maximum 0.5% inclusion area fraction. ENDURO® nitinol sold by SAES Smart Materials, Inc., New Hartford, New York, USA meets these specification. This nitinol is described in Yin, Weimin et al., “The Assessment of Physical and Mechanical Property Variability in a New Generation of Low Inclusion NiTi Alloy,” SMST 2022: Extended Abstracts from the International Conference on Shape Memory and Superelastic Technologies, May 16-20, 2022, Carlsbad, California, USA, Paper No: smst2022p0091, pp. 91-92; doi.org/10.31399/asm.cp.smst2022p0091. The ENDURO® ultra-low inclusion nitinol shows excellent fatigue performance and is compliant with the ASTM F2063-18 Standard Specification for Wrought Nickel-Titanium Shape Memory Alloys for Medical Devices and Surgical Implants.

In some embodiments, the nitinol used for the metal core is ultra-pure nitinol. The ultra-pure nitinol has the following approximate composition:

| Element | [wt. %] |
|---|---|
| Ni | 56.02 |
| C | 0.0258 |
| Co | 0.0001 |
| Cu | 0.0001 |
| Cr | 0.0017 |
| H | <0.0050 |
| Fe | 0.009 |
| Nb | 0.0001 |
| N | 0.0012 |
| O | 0.022 |
| Ti | (balance) |

The amounts of the trace elements (C, Co, Cu, Cr, H, Fe, Nb, N, and O) can vary plus-or-minus about 20% from the values shown (e.g., Co content can be between about 0.00008 to about 0.00012 weight percent), with corresponding adjustments to the Ni and/or Ti content to account for the variation in trace element content. The amounts of Ni and/or Ti can vary by about 0.05% of the amounts shown (e.g., Ni content can vary from about 55.99 to about 56.05 weight percent). ENDURO® nitinol sold by SAES Smart Materials, Inc., New Hartford, New York, USA meets these specifications.

In some embodiments, the nitinol used for the metal core is ultra-low inclusion, ultra pure nitinol, where the nitinol meets the criteria to be ultra-low inclusion nitinol and also meets the criteria to be ultra-pure nitinol. ENDURO® nitinol sold by SAES Smart Materials, Inc., New Hartford, New York, USA meets these specifications.

### Manufacturing Metal Cores

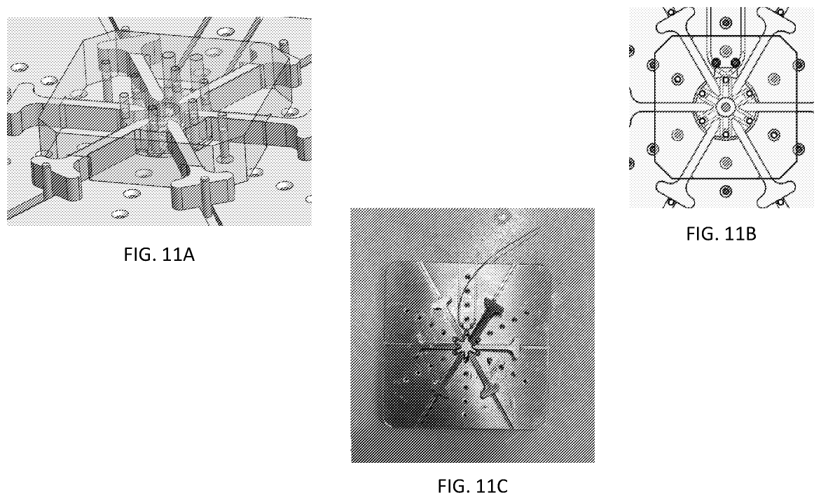

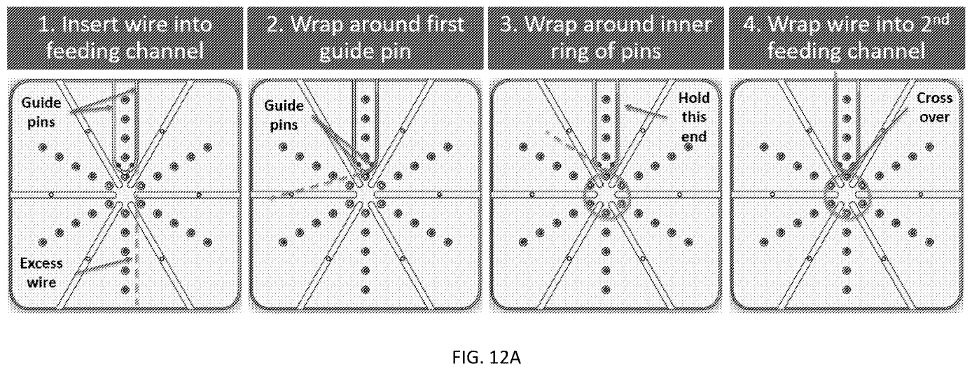

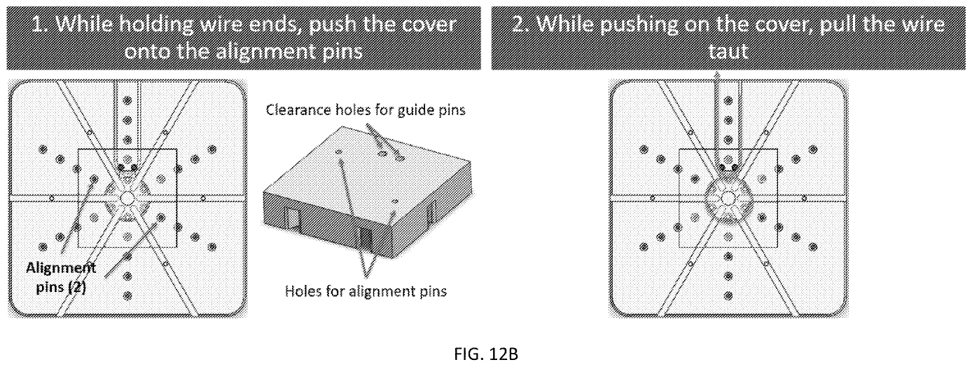

FIGS. 11A-11C show a metal wire core being formed using a jig. FIG. 11A shows a perspective view of a wire wrapped in to a stellate shape on a jig, according to some embodiments. FIG. 11B shows a wire wrapped into a stellate shape on a jig, according to some embodiments. FIG. 11C shows an image of a wire wrapped into a stellate shape on a jig, according to some embodiments. A jig can help improve the consistency of metal wire cores, and can help ensure that the metal wire core is symmetrical.

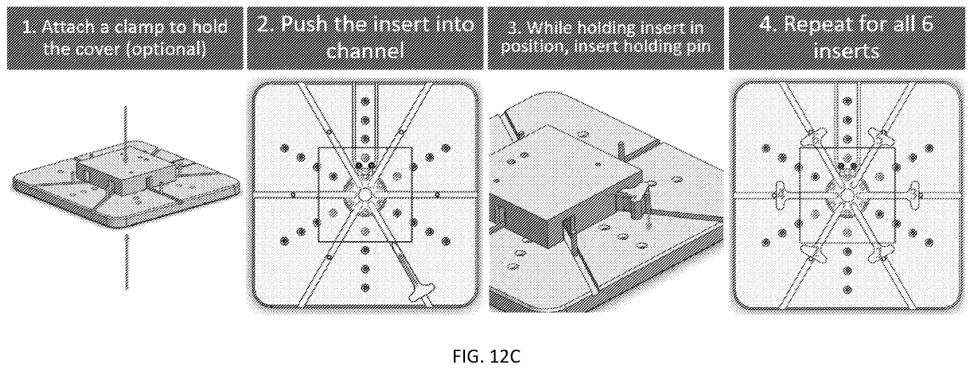

FIGS. 12A-12C show a process for forming a metal wire core using a jig, such as the jig depicted in FIGS. 11A-11C. Specifically, FIG. 12A shows the steps for wrapping the wire around pins of the jig to form the stellate shaped metal wire core. At step 1, a user will guide the wire into a feeding channel. At step 2, a user will wrap the wire around the first guide pin. The wire should be kept outside of the guide pin, but above the inner ring of pins. As step 3, a user can wrap the wire around the inner ring of pins. At step 4, a user will wrap the wire into a second feeding channel, such that the wire will cross over itself near the top pin. The wire should be kept to the outside of the guide pin.

Once the wire is wrapped according to the steps of FIG. 12A, a cover can be attached to the jig and on top of the wrapped wire. FIG. 12B shows the steps for attaching the cover. At step 1, a user can, while holding the wire ends, push the cover onto the alignment pins. The cover should be kept as parallel to the plate/jig as possible. At step 2, a user can pull the wire taut to ensure a properly formed metal wire core. The wire is sufficiently taut when both ends of the wire sit nicely in the feeding channels, and the cover is completely flush with the plate/jig.

Once the wire is taut and the cover is properly attached according to the steps of FIG. 12B, the stellate shape can be formed. FIG. 12C shows how inserts may be applied to form the stellate shape of the metal wire core. At step 1, a user can optionally attach a clamp to hold the cover to the plate/jig. At step 2, a user can push an insert into a radially-extending channel. At step 3, a user can insert the holding pin while holding the inserted insert in place. At step 4, a user can repeat steps 2 and 3 for the remaining inserts and holding pins.

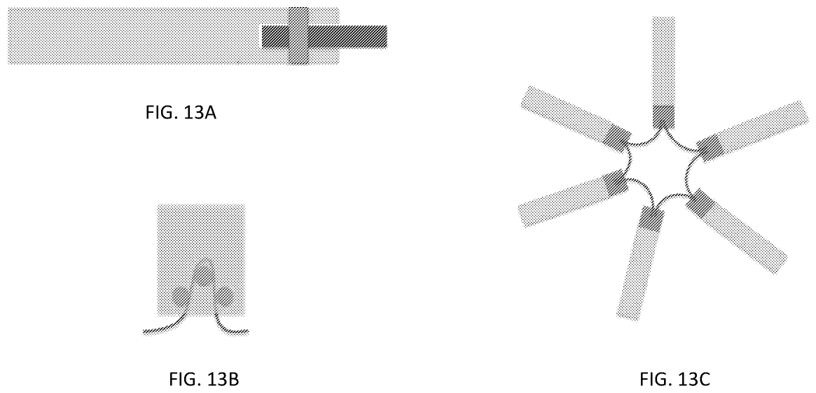

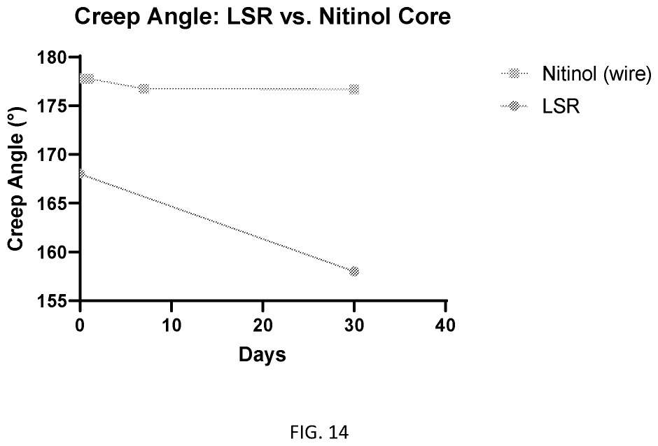

FIGS. 13A-13C show various configurations for affixing an arm of a gastric residence system to a metal core. Specifically, FIG. 13A shows a side view of FIG. 13B. FIG. 13B shows a slotted arm with pin, in which the arm mount of a metal wire core can hook around the pin of the allotted arm. FIG. 13C shows a metal core having pointed ends.

Several of the cores as illustrated, for example, in FIG. 6A or in FIG. 13C, show metal cores where the arm mounts are covered by polymeric linkers and/or elongate arms, but the innermost part of the metal cores are exposed. In some embodiments, the metal cores can be used in gastric residence systems with the innermost portion of the metal cores exposed. In some embodiments, the metal cores can be coated with a thin polymeric layer, particularly using a polymer that is stable and unreactive in a gastric environment, such as polytetrafluoroethylene (PTFE). The entire metal core, including the arm mounts, can be covered with the thin polymeric layer, or alternatively, the portion of the metal core that is not covered by the polymeric linkers and/or elongate arms (the innermost portion of the metal core) can be covered with the thin polymeric layer. The thin polymeric layer can be about 1 micron to about 100 microns in thickness, such as about 1 micron to about 75 microns, about 1 micron to about 50 microns, about 1 micron to about 40 microns, about 1 micron to about 30 microns, about 1 micron to about 25 microns, about 1 micron to about 20 microns, about 1 micron to about 10 microns, about 10 microns to about 20 microns, about 10 microns to about 30 microns, or about 20 microns to about 30 microns.

In some embodiments, the portion of the metal core that is not covered by the polymeric linkers and/or elongate arms can be covered by a thick polymeric layer, of approximately the same thickness and cross-sectional shape as the polymeric linkers on the arm mounts of the metal core, for example, within about 20%, about 10%, about 5%, or about 1% of the thickness of the polymeric linkers on the arm mounts of the metal core.

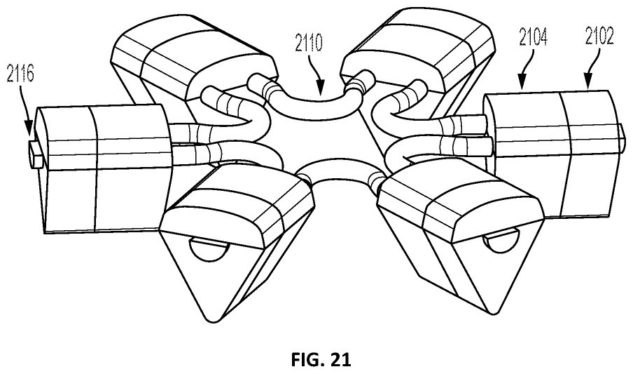

FIG. 21 shows an embodiment of a wire metal core with linkers installed on the arm mounts. Wire metal core 2110 has an inner linker 2104, and an outer linker 2102, where “inner” refers to a component closer to the center of the core and “outer” refers to a component further from the center of the core. The outer linker 2102 can optionally have a nub 2116, which can serve as a guide when further portions of the gastric residence system are connected to linker 2102.

The inner linker 2104 can be made of various materials, such as polycarbonate or polycaprolactone. Polycarbonate (medical grade) is particularly useful due to its strength and manufacturing convenience; polycarbonate made from Bisphenol A is typically used. The outer linker 2102 typically comprises polycaprolactone, which can be laser-welded or otherwise joined to elongate arms or segments making up elongate arms of the gastric residence system. In some embodiments, the outer linker can comprise the linker formulations disclosed in WO 2021/092487, incorporated herein by reference in its entirety, such as the formulations listed in Tables 3-7 and Tables 9-11 of WO 2021/092487; preferably, linker formulations which can be injection-molded are used. Blends of polycaprolactone with thermoplastic polyurethane (TPU) can be used. In some embodiments, the outer linker can comprise the linker formulations disclosed in WO 2022/159529 or WO 2023/141524, both of which are incorporated herein by reference in their entirety, such as the following linker formulations (the amounts are given in approximate weight percent, with the understanding that when ranges are provided, the amounts are chosen so as to add up to 100%):

| Matrix | Formu- | Formu- | Formu- | Formu- |
|---|---|---|---|---|
| Component | lation 1 | lation 2 | lation 3 | lation 4 |
| PCL (viscosity | 40-50 | 43-47 | 44.95 | 45 |
| midpoint 1.7 dl/g) | | | | |
| PDLG5004A | 30-40 | 33-37 | 35 | 35 |
| PDLG5004 | 10-25 | 15-20 | 18 | 18 |
| PEO(100k) | 0.5-5 | 1-3 | 2 | 2 |
| coloring (optional) | 0.005-0.2 | 0.01-0.1 | 0.05 (e.g. | 0 |
| | | | E172) | |

| | Formu- | Formu- | Formu- | Formu- |
|---|---|---|---|---|
| Matrix Component | lation 1 | lation 2 | lation 3 | lation 4 |
| PCL (viscosity | 40-50 | 43-47 | 44.95 | 45 |
| midpoint 1.2 dl/g) | | | | |
| PDLG5004A | 30-40 | 33-37 | 35 | 35 |
| PDLG5004 | 10-25 | 15-20 | 18 | 18 |
| PEO(100k) | 0.5-5 | 1-3 | 2 | 2 |
| coloring (optional) | 0.005-0.2 | 0.01-0.1 | 0.05 (e.g. | 0 |
| | | | E172) | |

| | Formu- | Formu- | Formu- | Formu- |
|---|---|---|---|---|
| Matrix Component | lation 1 | lation 2 | lation 3 | lation 4 |
| PCL (viscosity | 40-50 | 43-47 | 44.95 | 45 |
| midpoint 1.2 dl/g) | | | | |
| PDLG5004A | 48-58 | 51-55 | 53 | 53 |
| PDLG5004 | 0-5 | 0-2 | 0 | 0 |
| PEO(100k) | 0.5-5 | 1-3 | 2 | 2 |
| coloring (optional) | 0.005-0.2 | 0.01-0.1 | 0.05 | 0 |

| | Formu- | Formu- | Formu- | Formu- |
|---|---|---|---|---|
| Matrix Component | lation 1 | lation 2 | lation 3 | lation 4 |
| PCL (viscosity | 40-50 | 43-47 | 44.95 | 45 |
| midpoint 1.2 dl/g) | | | | |
| PDLG5002A | 48-58 | 51-55 | 53 | 53 |
| PDLG5002 | 0-5 | 0-2 | 0 | 0 |
| PEO(100k) | 0.5-5 | 1-3 | 2 | 2 |
| coloring (optional) | 0.005-0.2 | 0.01-0.1 | 0.05 | 0 |

The optional coloring can be iron oxide, such as E172 iron oxide. PCL=polycaprolactone; PDLG 5004=GMP grade copolymer of DL-lactide and glycolide (50/50 molar ratio) with inherent viscosity midpoint of 0.4 dl/g; PDLG 5004A=acid terminated GMP grade copolymer of DL-lactide and glycolide (50/50 molar ratio) with inherent viscosity midpoint of 0.4 dl/g; PDLG 5002=GMP grade copolymer of DL-lactide and glycolide (50/50 molar ratio) with inherent viscosity midpoint of 0.2 dl/g; PDLG 5002A=acid terminated GMP grade copolymer of DL-lactide and glycolide (50/50 molar ratio) with inherent viscosity midpoint of 0.2 dl/g; PEO=polyethylene oxide.

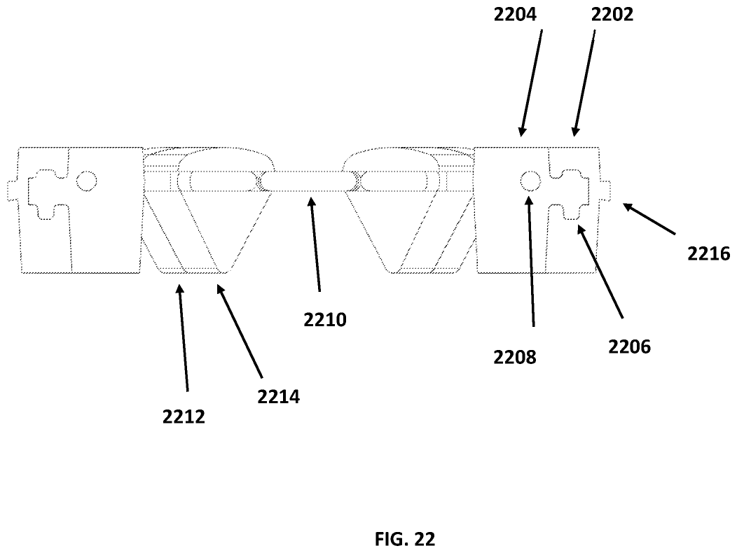

FIG. 22 shows a cross-sectional view of the wire metal core of FIG. 21, showing wire metal core 2210, inner linker 2204, outer linker 2202, and nub 2216. Feature 2208 shows the wire as it passes through inner linker 2204. Feature 2206 shows a projection of inner linker 2204 which extends into outer linker 2202. Outer linker 2202 can be overmolded onto inner linker 2204 in order to enclose the projection 2206 of inner linker 2204. Overmolding the outer linker 2202 such that it encloses the projection 2206 of inner linker 2204 strengthens the linkage between inner linker 2204 and outer linker 2202. Additional linker assemblies are shown, for example, inner linker 2214 and outer linker 2212 on a different arm mount, and additional inner and outer linkers, not labeled.

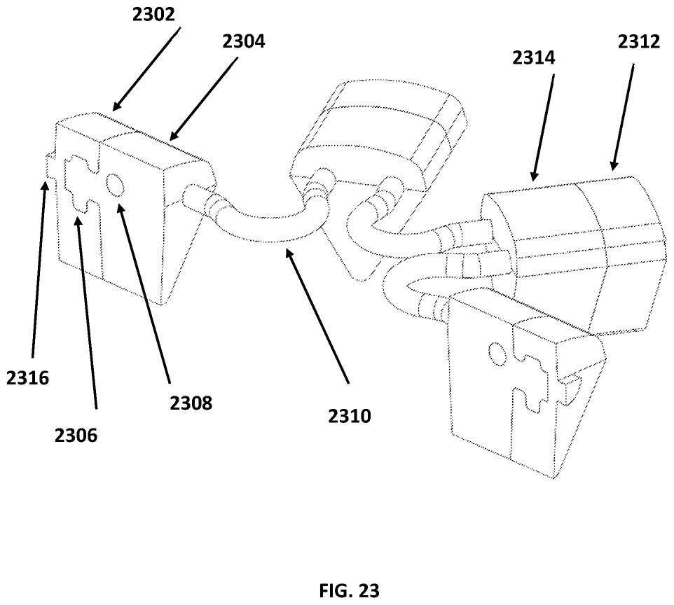

FIG. 23 shows another cross-sectional view of the wire metal core of FIG. 21. Wire metal core 2310, inner linker 2304, outer linker 2302, nub 2316, wire 2308 passing through inner linker 2304, projection 2306 of inner linker 2304 extending into outer linker 2302, and additional linker assemblies, including inner linker 2314 and outer linker 2312 on a different arm mount and additional unlabeled inner and outer linkers, are shown.

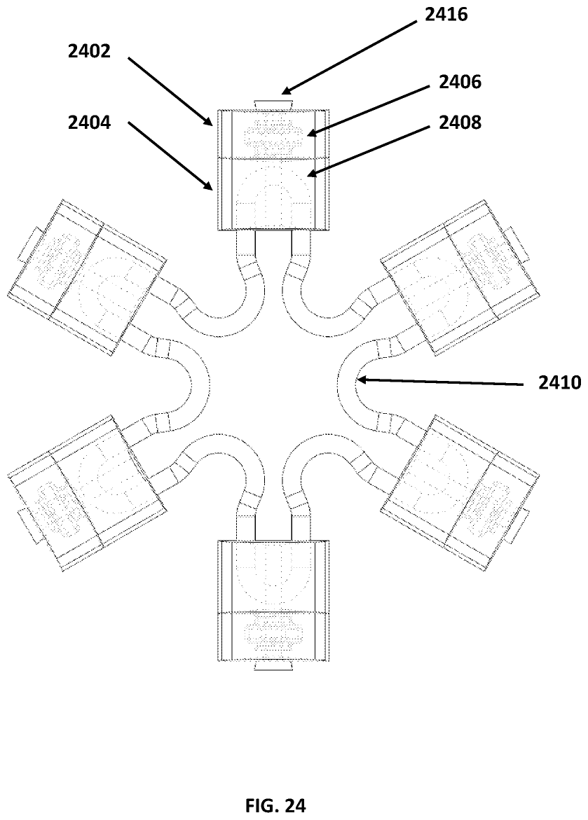

FIG. 24 shows another view of the nitinol wire core of FIG. 21 where the components covered by the polymeric linkers (i.e., the hidden components) are shown with dashed lines

### EXAMPLES

Example 1: FIG. 14 shows a the difference in creep angle (tested using the method explained below) between a nitinol metal core and a polymeric (liquid silicone rubber) core. As shown, the nitinol core wire largely maintains its creep angle over the course of 30 days, whereas the polymeric core's creep angle decreases steadily over the course of 30 days.

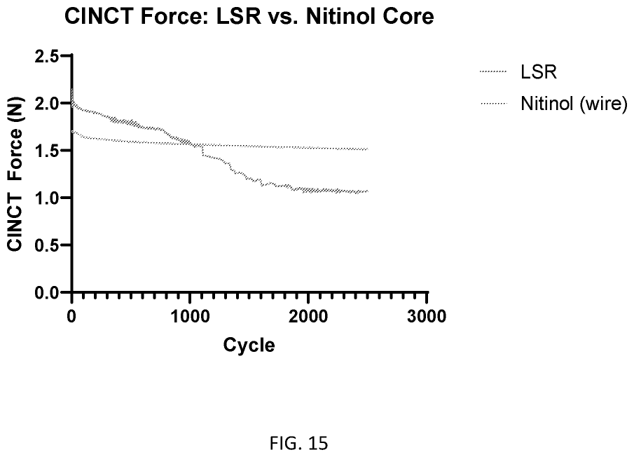

Example 2: FIG. 15 shows the cyclic incubated non-planar compression (CINCT) force for a nitinol core and a polymeric (i.e., liquid silicone rubber) core over the course of 2500 cycles. The CINCT force of the nitinol core remains relatively stable throughout the 2500 cycles, whereas the CINCT force of the polymeric core steadily decreases throughout the 2500 cycles.

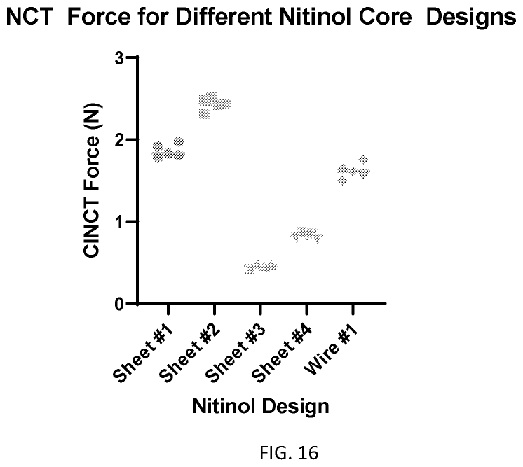

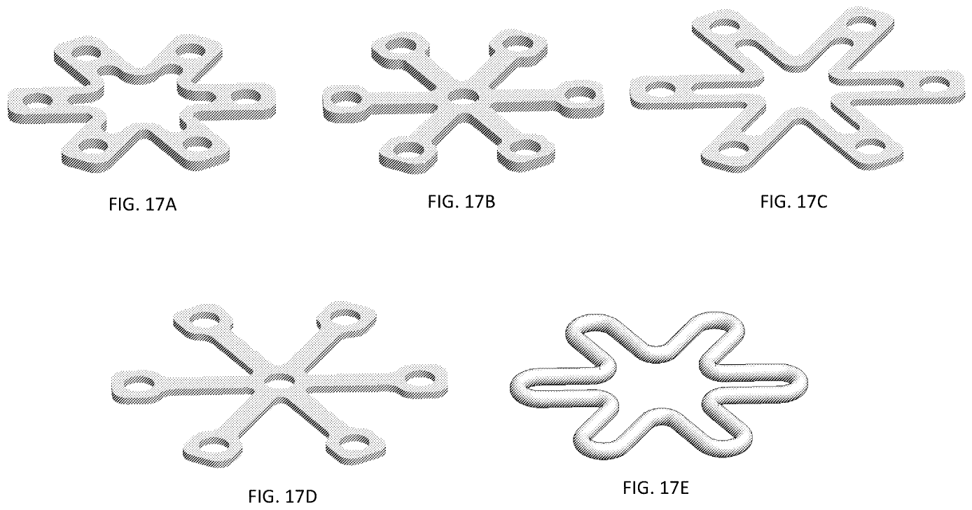

Example 3: FIG. 16 shows the cyclic incubated non-planar compression (CINCT) force for various nitinol core designs. Specifically, sheet 1 is depicted in FIG. 17A; sheet 2 is depicted in FIG. 1717B; sheet 3 is depicted in FIG. 17C, sheet 4 is depicted in FIG. 17D, and sheet 5 is depicted in FIG. 17E.

NCT Test: This test stands for “non-planar compression test”. During this test, the stellate is compressed between two blocks. Two arms contact each of the blocks, while the two remaining arms are unconstrained. The blocks apply stress to the core, causing it to deflect away from the plane of the original flat configuration. The test measures core stiffness. In some versions of the test, the compression is applied cyclically to measure fatigue properties of the core (called “CINCT”, or “cyclic incubated non-planar compression test”).

Creep Angle Test: In this test, a stellate is completely folded and stored into a capsule. At a certain time point, the stellate is removed from the capsule and springs back to a nearly-flat position. Using a special imaging stand, the angle formed by the two opposite arms with the flat base is measured. This test measures the ability of the core to elastically spring back to a flat position after being stored in a capsule.

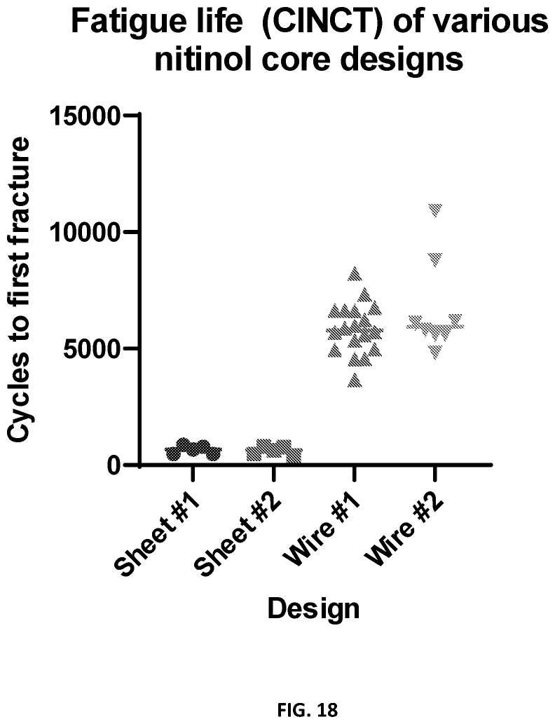

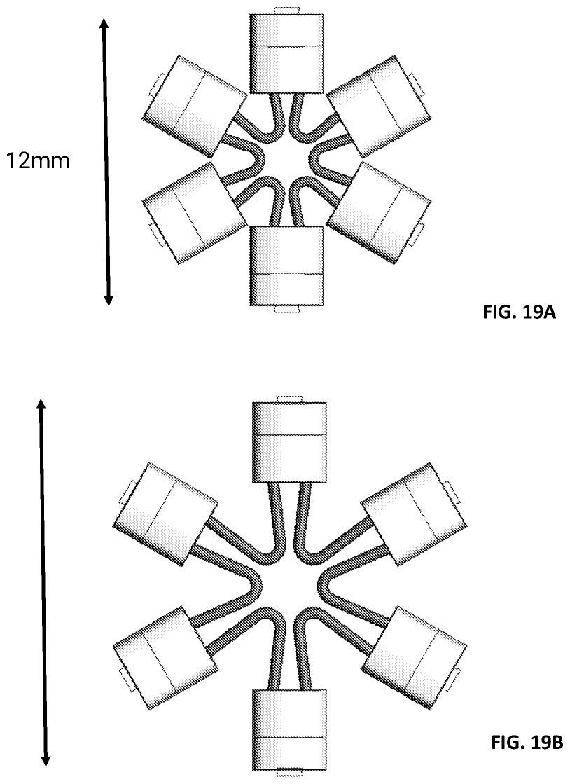

Example 4: FIG. 18 shows the performance of two nitinol core sheet designs (sheet #1 and sheet #2) and two nitinol core wire designs (wire #1 and wire #2) in the cyclic incubated non-planar compression test (CINCT). FIG. 17A and FIG. 17B show the geometries tested for Sheet #1 and Sheet #2, while FIG. 17E shows the wire form shape used for Wire #1 and Wire #2 results. The wire cores are able to go through significantly more cycles in the CINCT test than the sheet cores before fracture.

FIG. 19A and FIG. 19B show nitinol wire cores of different sizes, with linkers installed on the arm mounts. FIG. 19A shows a nitinol wire core which has an outer diameter (OD) of 12 mm from the outer edge of one linker to the outer edge of the opposing linker. The wire core itself has an outer diameter of 9 mm from the outer edge of one arm mount to the outer edge of the opposing arm mount. FIG. 19B shows a nitinol wire core which has an outer diameter (OD) of 15 mm from the outer edge of one linker to the outer edge of the opposing linker. The wire core itself has an outer diameter of 12 mm from the outer edge of one arm mount to the outer edge of the opposing arm mount. The longer arm mounts distribute the stress over a longer amount of material, thus lowering the local stress along the core.

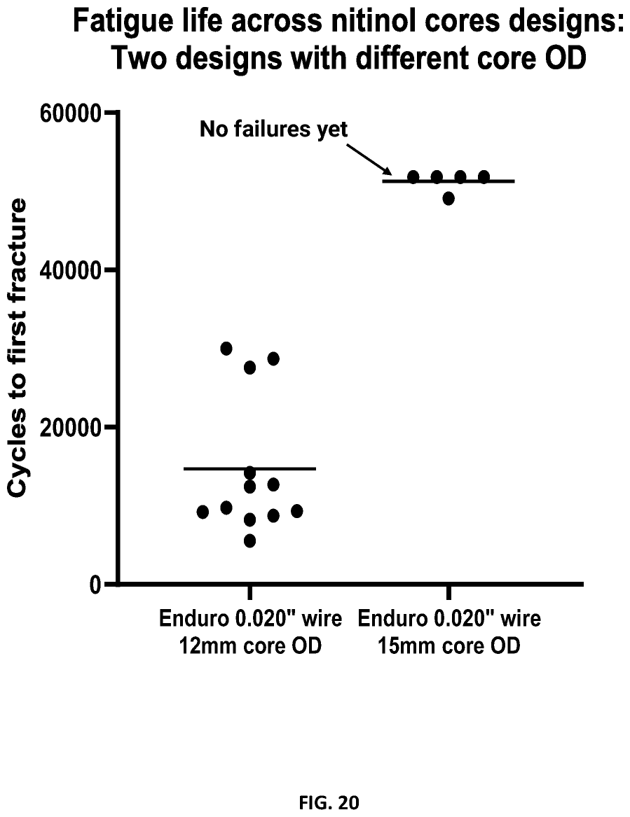

FIG. 20 shows cyclic incubated non-planar compression test (CINCT) results for the two cores shown in FIG. 19A and FIG. 19B. The 15 mm OD core of FIG. 19B, made out of a 0.020 inch (0.508 mm) diameter ENDURO® wire, was able to withstand more compression cycles without fracturing, as compared to the 12 mm OD core of FIG. 19A made from the same type of wire.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

Any of the systems, methods, techniques, and/or features disclosed herein may be combined, in whole or in part, with any other systems, methods, techniques, and/or features disclosed herein.

### EMBODIMENTS

Embodiment 1. A gastric residence system, comprising: a metal core, and a plurality of elongate arms, each elongate arm of the plurality of elongate arms comprising a proximal end connected to the metal core and a distal end, wherein the gastric residence system is configured to be folded into a compacted form by bending the metal core such that each arm mount of the plurality of arm mounts approach each other and the distal ends of the plurality of elongate arms approach each other, and wherein the metal of the metal core provides elasticity and shape memory to cause the gastric residence system to unfold into an uncompacted form and allow retention.

Embodiment 2. The gastric residence system of Embodiment 1, wherein the gastric residence system is configured to be stored in the compacted form for 30 days and then administered to a patient.

Embodiment 3. The gastric residence system of Embodiment 1 or 2, wherein the gastric residence system is configured to maintain the compacted form for 30 days and return to an uncompacted form such that the creep angle of the gastric residence form after the 30 days is within 3% of the creep angle before the 30 days.

Embodiment 4. The gastric residence system of any of Embodiments 1-3, wherein the metal core has a flat shape, such that a height and a width of the metal core is at least 10 times greater than a thickness of the metal core.

Embodiment 5. The gastric residence system of any of Embodiments 1-4, wherein a height of the metal core is the same as a width of the metal core.

Embodiment 6. The gastric residence system of any of Embodiments 1-5, wherein the metal core comprises a plurality of arm mounts, wherein each elongate arm of the gastric residence system is configured to attach to the metal core at an arm mount of the plurality of arm mounts.

Embodiment 7. The gastric residence system of any of Embodiments 1-6, wherein the metal core comprises nitinol.

Embodiment 8. The gastric residence system of any of Embodiments 1-7, wherein each elongate arm of the plurality of elongate arms is overmolded onto the metal core.

Embodiment 9. The gastric residence system of any of Embodiments 1-8, comprising a degradable linker attached to an arm mount of the metal core and an elongate arm attached to the degradable linker.

Embodiment 10. The gastric residence system of any of Embodiments 1-9, wherein the metal core is formed using metal wire.

Embodiment 11. The gastric residence system of any of Embodiments 1-10, wherein the metal core is formed with stamped metal sheets.

Embodiment 12. The gastric residence system of Embodiment 11, wherein the metal wire has a cross-section that is from 0.01 to 0.02 inches in diameter.

Embodiment 13. The gastric residence system of any of Embodiments 1-10, wherein the metal core is formed using flat sheets of metal.

Embodiment 14. The gastric residence system of any of Embodiments 1-13, wherein at least one elongate arm of the plurality of elongate arms comprises a therapeutic agent.

Embodiment 15. A core for a gastric residence system, comprising: a metal core having a plurality of arm mounts, each arm mount configured to receive an elongate arm of a gastric residence system, wherein the core is configured to bend into a compacted form such that each arm mount of the plurality of arm mounts approach each other and, when a plurality of elongate arms are attached to the metal core, the distal ends of the plurality of elongate arms approach each other, and wherein the metal of the metal core provides elasticity and shape memory to cause the metal core to unfold into an uncompacted form and, when a plurality of elongate arms are attached to the metal core, allow retention of the gastric residence system.

Embodiment 16. The core for a gastric residence system of Embodiment 15, wherein the metal core is configured to maintain the compacted form for 30 days and return to an uncompacted form such that the creep angle of the gastric residence form after the 30 days is within 3% of the creep angle before the 30 days.

Embodiment 17. The core for a gastric residence system of Embodiment 15 or 16, wherein the metal core has a flat shape, such that a height and a width of the metal core is at least 10 times greater than a thickness of the metal core.

Embodiment 18. The core for a gastric residence system of any of Embodiments 15-17, wherein a height of the metal core is the same as a width of the metal core.

Embodiment 19. The core of a gastric residence system of any of Embodiments 15-18, wherein the metal core is configured to maintain the compacted form for 30 days and return to an uncompacted form such that the creep angle of the metal core after the 30 days is within 3% of the creep angle before the 30 days.

Embodiment 20. The core for a gastric residence system of any of Embodiments 15-19, wherein the metal core comprises nitinol.

Embodiment 21. The core for a gastric residence system of any of Embodiments 15-20, wherein each arm mount is configured to receive an elongate arm that is overmolded onto the arm mount.

Embodiment 22. The core for a gastric residence system of any of Embodiments 15-21, wherein each arm mount is configured to receive a degradable linker and an elongate arm is attached to the degradable linker.

Embodiment 23. The core for a gastric residence system of any of Embodiments 15-22, wherein the metal core is formed using metal wire.

Embodiment 24. The core for a gastric residence system of Embodiment 21, wherein the metal wire has a cross-section that is from 0.01 to 0.02 inches in diameter.

Embodiment 25. The core for a gastric residence system of any of Embodiments 15-22, wherein the metal core is formed with stamped metal sheets.

Embodiment 26. The core for a gastric residence system of any of Embodiments 15-25, wherein at least one elongate arm of the plurality of elongate arms comprises a therapeutic agent.

Embodiment 27. A method of manufacturing a metal core for a gastric residence system, comprising:

wrapping metal wire using a jig to form a metal core comprising a plurality of arm mounts.

Embodiment 28. The method of Embodiment 27, wherein wrapping the metal wire comprises wrapping the metal wire around a plurality of pins of a jig to form a circular-shaped metal form.

Embodiment 29. The method of Embodiment 28, comprising advancing a plurality of inserts into the circular-shaped metal form to form a stellate-shaped metal form.

Embodiment 30. The method of Embodiment 29, comprising heating the stellate-shaped metal form to generate a stellate-shaped metal core for a gastric residence system.

Embodiment 31. The method of Embodiment 30, wherein heating the stellate-shaped metal form comprises heating the stellate-shaped metal form to 500 °C or greater.

Embodiment 32. The method of any of Embodiments 27-31, wherein the metal wire comprises nitinol.

Embodiment 33. The method of any of Embodiments 27-32, wherein the metal wire has a cross-section that is from 0.01 to 0.02 inches in diameter.

Embodiment 34. The method of any of Embodiments 27-33, wherein the two ends of the metal wire are joined together.

Embodiment 35. The method of any of Embodiments 27-33, wherein the two ends of the metal wire are not joined together.

Embodiment 36. The method of any of Embodiments 30-34, wherein the stellate-shaped metal form is quenched after heating.

Embodiment 37. The method of any of Embodiments 27-36, wherein the metal core has a flat shape such that a height and a width of the metal core is at least 10 times greater than a thickness of the metal core.

Embodiment 38. The method of Embodiment 37, wherein a height of the metal core is the same as a width of the metal core.

Embodiment 39. The method of any of Embodiments 27-38, wherein the metal core is configured to be bent into a compacted form and maintain the compacted form for 30 days and return to an uncompacted form such that the creep angle of the metal core after the 30 days is within 3% of the creep angle before the 30 days.

Embodiment 40. A method of manufacturing a metal core for a gastric residence system, comprising: stamping a metal sheet to form a metal core comprising a plurality of arm mounts.

Embodiment 41. The method of Embodiment 40, wherein the metal sheet comprises nitinol.

Embodiment 42. The method of Embodiment 40 or 41, wherein the metal core has a flat shape such that a height and a width of the metal core is at least 10 times greater than a thickness of the metal core.

Embodiment 43. The method of Embodiment 42, wherein a height of the metal core is the same as a width of the metal core.

Embodiment 44. The method of any of Embodiments 40-43, wherein the metal core is configured to be bent into a compacted form and maintain the compacted form for 30 days and return to an uncompacted form such that the creep angle of the metal core after the 30 days is within 3% of the creep angle before the 30 days.

1. A gastric residence system, comprising:
a metal core comprising a metal wire wrapped into a shape having a plurality of arm mounts, and
a plurality of elongate arms, each elongate arm of the plurality of elongate arms comprising a proximal end connected to an arm mount of the metal core and a distal end,
wherein the gastric residence system is configured to be folded into a compacted form by bending the metal core such that each arm mount of the plurality of arm mounts approach each other and the distal ends of the plurality of elongate arms approach each other, and
wherein the metal of the metal core provides elasticity and shape memory to cause the gastric residence system to unfold into an uncompacted form and allow retention.

2. The gastric residence system of claim 1, wherein the gastric residence system is configured to be stored in the compacted form for 30 days and then administered to a patient.

3. The gastric residence system of claim 1, wherein the gastric residence system is configured to maintain the compacted form for 30 days and return to an uncompacted form such that the creep angle of the gastric residence form after the 30 days is within 3% of the creep angle before the 30 days.

4. The gastric residence system of claim 1, wherein the metal core has a flat shape, such that a height and a width of the metal core is at least 10 times greater than a thickness of the metal core.

5. The gastric residence system of claim 1, wherein a height of the metal core is the same as a width of the metal core.

6. The gastric residence system of claim 1, wherein the metal core comprises a plurality of arm mounts, wherein each elongate arm of the gastric residence system is configured to attach to the metal core at an arm mount of the plurality of arm mounts.

7. The gastric residence system of claim 1, wherein the metal core comprises nitinol.

8. The gastric residence system of claim 7, wherein the nitinol comprises ultra-low inclusion nitinol.

9. The gastric residence system of claim 7, wherein the nitinol comprises ultra-pure nitinol.

10. The gastric residence system of claim 7, wherein the nitinol comprises ultra-low inclusion, ultra-pure nitinol.

11. The gastric residence system of claim 1, wherein each elongate arm of the plurality of elongate arms is overmolded onto the metal core.

12. The gastric residence system of claim 1, comprising a degradable linker attached to an arm mount of the metal core and an elongate arm attached to the degradable linker.

13. The gastric residence system of claim 1, comprising an inner linker and an outer linker attached to an arm mount of the metal core, and an elongate arm attached to the outer linker.

14. The gastric residence system of claim 13, wherein the inner linker comprises polycarbonate.

15. The gastric residence system of claim 13, wherein the outer linker comprises polycaprolactone.

16. The gastric residence system of claim 13, wherein a projection of the inner linker extends into the outer linker.

17-18. (canceled)

19. The gastric residence system of claim 1, wherein the metal wire has a cross-section that is from 0.01 to 0.02 inches in diameter.

20. (canceled)

21. The gastric residence system of claim 1, wherein at least one elongate arm of the plurality of elongate arms comprises a therapeutic agent.

22. A core for a gastric residence system, comprising:
a metal core comprising a metal wire wrapped into a shape having a plurality of arm mounts, each arm mount configured to receive an elongate arm of a gastric residence system,
wherein the core is configured to bend into a compacted form such that each arm mount of the plurality of arm mounts approach each other and, when a plurality of elongate arms are attached to the metal core, the distal ends of the plurality of elongate arms approach each other, and
wherein the metal of the metal core provides elasticity and shape memory to cause the metal core to unfold into an uncompacted form and, when a plurality of elongate arms are attached to the metal core, allow retention of the gastric residence system.

23-40. (canceled)

41. A method of manufacturing a metal core for a gastric residence system, comprising:
wrapping metal wire using a jig to form a metal core comprising a plurality of arm mounts.

42. The method of claim 41, wherein wrapping the metal wire comprises wrapping the metal wire around a plurality of pins of a jig to form a circular-shaped metal form.

43. The method of claim 42, comprising advancing a plurality of inserts into the circular-shaped metal form to form a stellate-shaped metal form.

44. The method of claim 43, comprising heating the stellate-shaped metal form to generate a stellate-shaped metal core for a gastric residence system.

45. The method of claim 44, wherein heating the stellate-shaped metal form comprises heating the stellate-shaped metal form to 500 °C or greater.

46-56. (canceled)

57. A method of manufacturing a metal core for a gastric residence system, comprising:
stamping a metal sheet to form a metal core comprising a plurality of arm mounts.

58-64. (canceled)

65. A gastric residence system, comprising:
a metal core comprising a stamped metal sheet having a plurality of arm mounts, and
a plurality of elongate arms, each elongate arm of the plurality of elongate arms comprising a proximal end connected to an arm mount of the metal core and a distal end,
wherein the gastric residence system is configured to be folded into a compacted form by bending the metal core such that each arm mount of the plurality of arm mounts approach each other and the distal ends of the plurality of elongate arms approach each other, and
wherein the metal of the metal core provides elasticity and shape memory to cause the gastric residence system to unfold into an uncompacted form and allow retention.

66. A core for a gastric residence system, comprising:
a metal core comprising a stamped metal sheet having a plurality of arm mounts, each arm mount configured to receive an elongate arm of a gastric residence system, wherein the core is configured to bend into a compacted form such that each arm mount of the plurality of arm mounts approach each other and, when a plurality of elongate arms are attached to the metal core, the distal ends of the plurality of elongate arms approach each other, and
wherein the metal of the metal core provides elasticity and shape memory to cause the metal core to unfold into an uncompacted form and, when a plurality of elongate arms are attached to the metal core, allow retention of the gastric residence system.
